(19) United States
(12) Patent Application Publication
(10) Pub. No.: US 2006/0241633 A1
(43) Pub. Date: Oct. 26, 2006

(54) Patella protector

(76) Inventors: Greg Stalcup, Columbia City, IN (US); Mark Bryant, Auburn, IN (US)

Correspondence Address: TAYLOR & AUST, P.C., 142 SOUTH MAIN STREET, P. O. BOX 560, AVILLA, IN, 46710, US

(21) Appl. No.: 11/115,007

(22) Filed: Apr. 26, 2005

(51) Int. Cl. A61F 5/00

(52) U.S. Cl. 606/86

(57) ABSTRACT

A patella protector for protecting a patella of a knee during surgery including a platform being sized and shaped to be slid under the patella. The platforms includes at least one surface. The patella protector further includes a web with a first end and a second end opposite the first end. The first end is connected to at least one surface of the platform. The web terminates in a gripping element at the second end.

### BACKGROUND OF THE INVENTION

1. Field of the Invention

The present invention relates to surgical instruments, and, more particularly, to orthopaedic surgical instruments.

2. Description of the Related Art

During total knee surgery it is common to make a longitudinal cut over the top of the patella and then resect the soft tissue to the sides. The patella is pulled to one side to expose the ends of the tibia and femur so that they may be cut and a knee component installed. Depending upon the type of knee component to be installed, as well as the quality of the patella, it is sometimes necessary to cut the bottom side of the patella to form a flat surface (as opposed to a slightly parabolic surface as is anatomically natural on the bottom side of the kneecap.) Conventionally, the kneecap is then pried to one side by using a rod or retractor to move the kneecap out of the way for the remaining surgical procedures. This prying action can damage the flat resected surface, and/or can even break the patella, which obviously creates undesired complications during and after surgery.

A patella preparation apparatus and method for the resection of a patella is known which includes a patella clamp with a frame, an upper handle affixed to the frame, and a lower handle mounted upon the frame for pivotal movement toward and away from the upper handle. A pair of laterally extending posts are carried by a bushing member, and an indicator in the form of a depth gauge on the bushing member includes a visible scale, for purpose of allowing a surgeon to determine, with precision, the thickness of the patella merely by observing an indicator in the form of the visible scale. The surgeon is therefore provided with a quick and accurate direct interoperative measurement which the surgeon uses to determine the amount of bone to be removed during subsequent resection of patella. Although this instrument is suitable for its intended purpose as an aid to the resection of the patella, it is not suited for protecting the patella after resection, and/or during subsequent procedures as, for example, the removal of the ends of the tibia and femur. The patella preparation apparatus as described above is simply too bulky and awkward for use as a patella protector as it will get in the way of subsequent procedures. Further, the patella preparation apparatus has no way of holding the patella except through clamping action which may damage or break the patella. Yet further, this known patella preparation apparatus is relatively complex in design, and therefore expensive to manufacture, and not particularly ergonomic in use.

A surgical retractor is known which is used as an upper abdominal retractor, and more particularly, as a self retaining upper abdominal retractor. This retractor includes a circular frame and multiple retractors which each have an arm which connects to the frame and a blade which contacts the soft tissue of the abdominal wall and viscera. At least one retractor includes a blade which is designed with two wings and a notch therebetween which can be positioned over the spinal promontory without damaging the same, and which also can avoid compressing the descending colon. Although this self retaining upper abdominal retractor has a utility in the retraction of the upper abdomen, it is totally unsuited to protecting the patella during surgery.

What is needed in the art is a patella protector which can protect the patella during knee surgery, and which is easily used during knee surgery without interrupting or obstructing other procedures which are part of the knee surgery.

### SUMMARY OF THE INVENTION

The present invention provides a patella protector which is easy to use and which protects the patella during knee surgery.

The invention comprises, in one form thereof, a patella protector for protecting a patella of a knee during surgery including a platform being sized and shaped to be slid under the patella. The platforms includes at least one surface. The patella protector further includes a web with a first end and a second end opposite the first end. The first end is connected to at least one surface of the platform. The web terminates in a gripping element at the second end.

The invention comprises, in another form thereof, a patella protector for protecting a patella of a knee during surgery including a platform and, in one embodiment, a curved handle attached to the platform.

The invention comprises, in another form thereof, a method of protecting a patella of a knee during surgery, including the steps of: providing a patella protector; sliding a surface of the patella protector between the patella and knee; and retracting the patella at least partially from a surgical field of the surgery using the patella protector.

The invention comprises, in another form thereof, a patella protector for protecting a patella of a knee during surgery including a platform being sized and shaped to be slid under the patella where the platform has a patella contacting surface with a non-slip surface.

An advantage of the present invention is that it protects the patella during knee surgery.

Another advantage of the present invention is that it is cost effective to manufacture.

Yet another advantage of the present invention is that it is ergonomic in use.

Yet another advantage of the present invention is that it protects damage to the patella which may occur during orthopaedic surgery while using a saw, mill or the like to cut the ends of the tibia and femur.

Yet another advantage of the present invention is that it does not interrupt or obstruct other procedures which are part of the knee surgery.

Yet another advantage of the present invention is that it provides a non-slip surface for the patella.

Yet another advantage of the present invention is that it does not require a clamping of the patella in the patella protector.

Yet another advantage of the present invention is that it is self-retaining.

Yet another advantage of the present invention is that it covers the resected patella.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 is a perspective view of a first embodiment of the present invention;

FIG. 2 is a side view of the embodiment of FIG. 1;

FIG. 3 is a enlarged fragmentary detail side view of the non-slip surface of the embodiment of FIG. 1;

FIG. 4 is a perspective view of a second embodiment of the present invention; and

FIG. 5 is a side view of a third embodiment of the present invention.

Corresponding reference characters indicate corresponding parts throughout the several views. The exemplifications set out herein illustrate one preferred embodiment of the invention, in one form, and such exemplifications are not to be construed as limiting the scope of the invention in any manner.

### DETAILED DESCRIPTION OF THE INVENTION

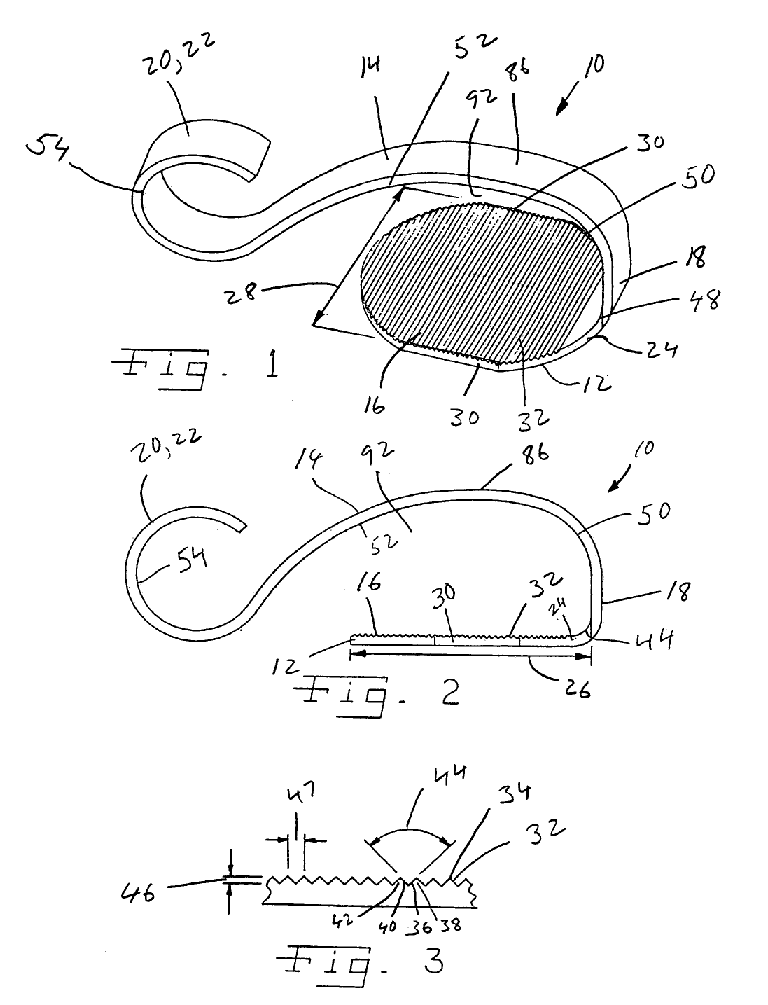

Referring now to the drawings, and more particularly to FIG. 1, there is shown a patella protector 10, for protecting a patella of a knee during surgery, which generally includes a platform 12 and a web 14 connected to platform 12. Platform 12 is sized and shaped to be slid under the patella. Platform 12 includes at least one surface 16. Web 14 includes a first end 18 and a second end 20 opposite first end 18. First end 18 is connected to at least one surface 16 of platform 12. Web 14 terminates in a gripping element 22 at second end 20.

In the embodiment shown in FIG. 1, first end 18 is connected transversely to platform 12. At least one surface 16 can comprise an edge 24 of platform 12 such that web 14 is connected to edge 24. Platform 12 can have approximately the form of a plate. Platform 12 can be an approximately flat plate although other contours are possible which include a platform 12 with curvature about at least one axis. Such curvature can be, but is not limited to, circular, parabolic, elliptical, some combination thereof, or other complex curvatures.

Surface 16 includes a patella contacting surface with a first width 26, and a second width 28 defining at least one truncated edge 30 of patella contacting surface 16. In one embodiment, first width 26 can be approximately 1.750 inches and second width 28 can be approximately 1.650 inches.

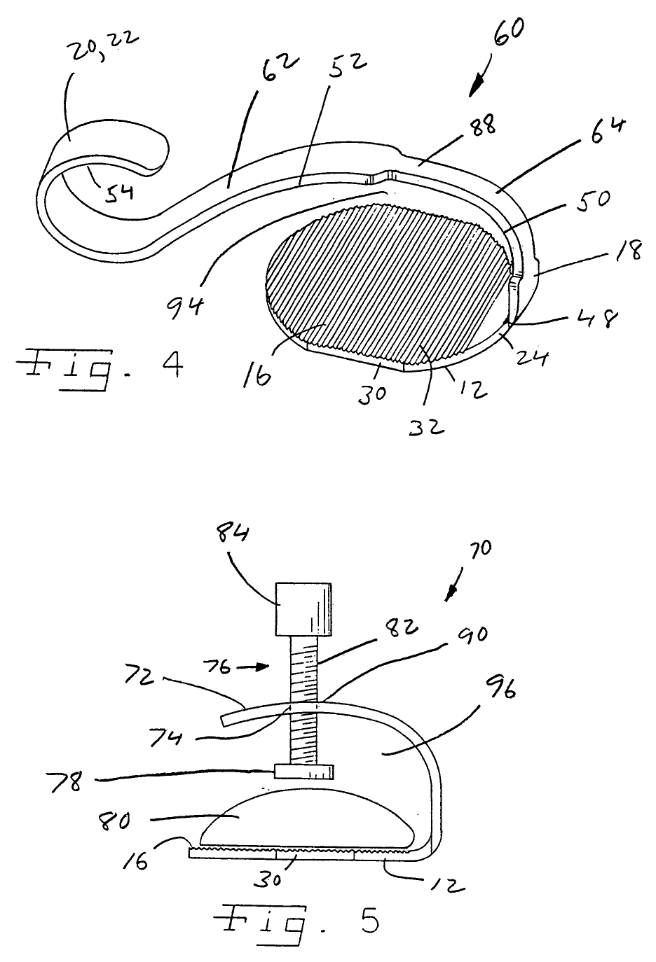

Patella contacting surface 16 can include a non-slip surface which is shown as a serrated surface. The serrated surface includes a plurality of approximately triangular ridges 32 (FIG. 3). Each of the plurality of approximately triangular ridges 32 includes a vertex 34 with a radius. In one embodiment, each radius is at least 0.005 inches. A first side 36 of a first triangular ridge 38 intersects a second side 40 of a second triangular ridge 42, and an angle 44 between first side 36 and second side 40 subtends approximately 90°, although other angles are possible. In general, angle 44 can be between 0° and 180°. A height 46 of a triangular ridge 32 can be approximately between 0.020 and 0.025 inches. A distance between adjoining vertices 34 can be approximately 0.040 inches, for example. Patella contacting surface 16 can include an approximately periodic, regular serrated surface as shown, or alternatively, can include a non-periodic and/or irregular non-slip surface.

In the embodiments of FIGS. 1 and 4, gripping element 22 is a curved handle. More specifically, web 14 can include a first radius 48 which extends web 14 from platform 12, a second radius 50 which extends web 14 across platform 12, and a third radius 52 which extends web 14 towards platform 12. Web 14 is curved in a similar orientation through first radius 48, second radius 50 and third radius 52. Web 14 can include a fourth radius 54 which is curved in an opposite orientation than the previously described similar orientation, and gripping element 22 can be included within fourth radius 54. In one embodiment, first radius 48 is approximately between 0.100 and 0.200 inches, second radius 50 is approximately between 0.439 and 0.639 inches, third radius 52 is approximately between 1.793 and 1.993 inches and fourth radius 54 is approximately between 0.375 and 0.475 inches.

In the embodiment of FIG. 4, patella protector 60 has web 62 with a reduced width section 64 which has at least the advantages of reducing the weight of patella protector 60, and which can allow better visibility of the patella. Other features and structure of patella protector 60 are similar to patella protector 10.

In the embodiment of FIG. 5, patella protector 70 has web 72 with a threaded hole 74 at a second end of web 72 through which gripping element or positive locking mechanism 76 is engaged. Positive locking mechanism 76 can include a engaging end 78 for engaging the patella 80, a threaded portion or shank 82 and an actuating end 84 which can include a knurled knob, for example. Other features and structure of patella protector 70 are similar to patella protector 10. Although all of patella protectors 10, 60 and 70 are self-retaining, patella protector 70 provides positive locking onto patella 80 by tightening locking mechanism 76 onto patella 80.

Each of patella protectors 10, 60 and 70 include a platform 12 and a curved handle 14, 62 and 72, respectively, attached to platform 12. A portion 86, 88, 90, of curved handles 14, 62, 72, respectively, are generally coplanar with platform 12 and define a patella receiving slot 92, 94, 96, respectively, therebetween. As shown particularly in FIGS. 1, 2 and 4, handles 14 and 62 have a generally S-shaped cross-sectional configuration.

In use, the present invention provides a method of protecting a patella of a knee during surgery, including the steps of: providing a patella protector 10, 60 or 70; sliding a surface 16 of patella protector 10, 60 or 70 between patella 80 and knee; and retracting patella 80 at least partially from a surgical field of the surgery using patella protector 10, 60 or 70. The method can further include the step of retaining patella 80 within patella protector 10, 60 or 70 during an operation of the surgery.

While this invention has been described as having a preferred design, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A patella protector for protecting a patella of a knee during surgery, comprising:
a platform being sized and shaped to be slid under the patella, said platform including at least one surface; and
a web including a first end and a second end opposite said first end, said first end being connected to at least one said surface of said platform, said web terminating in a gripping element at said second end.

2. The patella protector of claim 1, wherein said first end is connected transversely to said platform.

3. The patella protector of claim 1, wherein said at least one surface comprises an edge of said platform.

4. The patella protector of claim 1, wherein said platform approximately has the form of a plate.

5. The patella protector of claim 4, wherein said plate is approximately flat.

6. The patella protector of claim 1, wherein said at least one surface includes a patella contacting surface including a first width, and a second width defining at least one truncated edge of said patella contacting surface.

7. The patella protector of claim 1, wherein said at least one surface includes a patella contacting surface, and said patella contacting surface has a non-slip surface.

8. The patella protector of claim 7, wherein said non-slip surface is a serrated surface.

9. The patella protector of claim 8, wherein said serrated surface includes a plurality of approximately triangular ridges.

10. The patella protector of claim 9, wherein each of said plurality of approximately triangular ridges includes a vertex with a radius.

11. The patella protector of claim 10, wherein each said radius is at least 0.005 inches.

12. The patella protector of claim 9, further including a first side of a first triangular ridge intersecting a second side of a second triangular ridge, an angle between said first side and said second side subtending approximately 90°.

13. The patella protector of claim 1, wherein said gripping element is a curved handle.

14. The patella protector of claim 1, wherein said gripping element is a screw threadingly engaged with said second end for gripping the patella between said screw and said platform.

15. A patella protector for protecting a patella of a knee during surgery, comprising:
a platform configured to be slid under the patella; and
a curved handle attached to said platform.

16. The patella protector of claim 15, further including a portion of said curved handle lying generally coplanar with said platform and defining a patella receiving slot therebetween.

17. The patella protector of claim 15, wherein said handle has a generally S-shaped cross-sectional configuration.

18. A method of protecting a patella of a knee during surgery, comprising the steps of:
providing a patella protector;
sliding a surface of said patella protector between the patella and the knee; and
retracting the patella at least partially from a surgical field of the surgery using said patella protector.

19. The method of claim 18, wherein said patella protector includes a platform sized and shaped to be slid under the patella, said platform including at least one surface, said patella protector further includes a web with a first end and a second end opposite said first end, said first end connected to at least one said surface of said platform, said web terminates in a gripping element at said second end.

20. The method of claim 18, further including the step of retaining the patella within said patella protector during an operation of the surgery.

21. A patella protector for protecting a patella of a knee during surgery, comprising:
a platform being sized and shaped to be slid under the patella, said platform including a patella contacting surface having a non-slip surface.
